(19) United States
(12) Patent Application Publication
(10) Pub. No.: US 2022/0168210 A1
(43) Pub. Date: Jun. 2, 2022

(54) HIGH FOAMING CLEANSING COMPOSITION

(71) Applicant: L'OREAL, Paris (FR)

(72) Inventors: Hungta LIN, Teaneck, NJ (US); Ryuji HARA, Springfield, NJ (US); Siliu TAN, Westfield, NJ (US)

(73) Assignee: L'OREAL, Paris (FR)

(21) Appl. No.: 17/107,432

(22) Filed: Nov. 30, 2020

(51) Int. Cl. A61K 8/87; A61K 8/06; A61K 8/898; A61K 8/86; A61K 8/92; A61K 8/46; A61K 8/31; A61K 8/37; A61Q 1/14

(52) U.S. Cl. CPC A61K 8/87; A61K 8/062; A61K 8/068; A61K 8/898; A61K 8/86; A61K 2800/28; A61K 8/463; A61K 8/31; A61K 8/37; A61Q 1/14; A61K 8/922

(57) ABSTRACT

A cleansing composition includes at least one water-based polyurethane, at least one silicone oil-in-water microemulsion, and at least one oil or ester. The cleansing composition is clear and includes a pleasant skin-feel when applied, demonstrating high foam production in formulations that include up to about 50 wt % oil and high capacity for removal of oily pigments.

### FIELD OF TECHNOLOGY

The present disclosure is directed to cleansing compositions. More specifically, the present disclosure is directed to a high-foaming cleanser composition with high pigment removal capacity, the composition having two phases and including a polyurethane, a silicone oil-in-water microemulsion, and an oil or ester.

### BACKGROUND

Consumers of cosmetic cleansing compositions often seek products that have a pleasing aesthetic property of foaming which is perceived by a consumer as evidence of effective cleansing capacity. For use as a make-up remover, such cleansers are ideally capable of effectively removing make-up that includes a high amount of pigment. There are several examples in the art that describe products that have one or the other of these desirable properties that include high-foaming and pigment removal.

Conventional cleansers that include oil and/or esters for emollient properties and for oil removal from skin and hair typically employ surfactants that can provide pleasing foaming. But these compositions typically are oil in water emulsions which can effectively prevent the oil phase from contacting and removing makeup.

Conventional make-up removal products that contain a high oil and/or ester content that is suitable for removal of high pigment makeup do not exhibit any meaningful foaming property. There is a need for a cleansing composition that provides the desired foaming properties that consumers strongly prefer combined with strong capacity for removal of oily-based pigmented make-ups, which also provide other beneficial and efficacy properties that consumers seek, including pleasant skin-feel and gentle emollient and cleansing benefits.

The inventors hereof have provided a clear composition that combines the benefits of conventional cleansers and makeup removers into a single formulation with desirable properties that consumers seek. Inclusion of the polyurethane and silicone oil-in-water microemulsion components permit the high oil/ester component to contact the keratinous substrate and achieve high pigment removal capacity while at the same time conferring a pleasing high-foaming property.

### BRIEF SUMMARY

In an exemplary embodiment, a cleansing composition includes at least one polyurethane, at least one silicone oil-in-water microemulsion, and at least one oil or ester. The cleansing composition is essentially free of emulsifiers.

Other features and advantages of the present disclosure will be apparent from the following more detailed description of the preferred embodiment which illustrates, by way of example, the principles of the disclosure.

### DETAILED DESCRIPTION

According to the disclosure, a cleansing composition is provided that includes at least one polyurethane, at least one silicone oil-in-water microemulsion, and at least one oil or ester. The cleansing composition is clear and includes a pleasant skin-feel when applied, demonstrating unexpectedly high foam production in formulations that include up to about 50 wt % oil. The composition also provides gentle emollient and cleansing benefits including a surprising and unexpectedly high capacity for removal of oily pigments. Current personal care products provide only one of the features of high-foaming and high pigment removal. Typical “cleansers” provide high foaming but are not particularly efficient at removing heavy, oily pigments such as are found in lipstick, mascara and other make-up compositions. Typical “makeup removers” provide acceptable pigment removal capacity, but do not include foaming. Consumers express a desire for products that have both properties. This disclosure provides such a composition which includes the high foaming benefits seen in typical cleansers and high pigment removal capacity seen in typical makeup removers, combined in a single composition.

As used herein, the term “stability,” with respect to a cleansing composition, refers to the cleansing composition remaining in a mixed high-viscosity state and not separating, such as not phase separating. Such properties may be achieved even with the inclusion of high amounts of oils and/or esters, which typically change the surface tension and reduce or prevent foaming of a composition. In some embodiments, a cleansing composition has a high viscosity while also containing a high concentration of oils and/or esters, which tend to reduce the viscosity of a composition. Such properties may be achieved even with the inclusion of high amounts of silicones. In some embodiments, a cleansing composition has a high viscosity while also containing a high concentration of silicones, which would typically tend to reduce the viscosity of a composition.

Stability is evidenced by one or both of direct measurement of viscosity or visual inspection. For purposes hereof, a composition that demonstrates physical stability and does not become loose or flowing, as evidenced by visual inspection, is stable. In various embodiments, an inventive composition remains stable at temperatures in the range from about 5° C. to about 45° C., over a time period of at least two months, or at least three months, or at least four months, or at least six months, or any value, range, or sub-range therebetween. In some embodiments, a cleansing composition is stable at 25° C. for at least three months, or at least four months, or at least six months, or at least nine months, or at least 12 months, or any value, range, or sub-range therebetween. In some embodiments, a cleansing composition is stable at 45° C. for at least one month, or at least two months, or at least three months, or at least four months, or at least six months, or any value, range, or sub-range therebetween. As disclosed and exemplified herein, a cleansing composition is stable at 45° C. for at least about three months or more.

As used herein with respect to the foaming property of cleansing formulations, including the inventive cleansing composition, the term “Foaming Capacity” means and refers to the capacity of a composition (inventive or comparative) to develop a foam upon rubbing or agitation that would be associated with applying and gently scrubbing skin or other keratinous substrates with the formulation. Reference is made to the testing method for establishing Foaming Capacity employing equipment that is well known in the art, namely analysis using a Krauss DFA100™ Dynamic Foam Analyzer, which provides a relative measure of achieved foam height upon agitation in a sample having a fixed volume. Foaming Capacity, as used herein relative to foaming on a keratinous substrate, such as facial skin, is based on a scale from low to high, where the scale generally corelates with foaming using a dynamic foam analyzer to provide: low<1=220 ml foam), middle (˜225-240 ml), to high (>1=250 ml). Thus, the term “high-foaming,” with respect to a cleansing composition, refers to a composition that has a high foaming capacity.

In exemplary embodiments, the cleansing composition includes an emulsion. In some embodiments, the cleansing composition is transparent.

In some embodiments, the cleansing composition is a skin cleanser.

In some embodiments, the cleansing composition is a shampoo.

In some embodiments, the cleansing composition is a body wash.

In some embodiments, the cleansing composition is a make-up remover.

Polyurethane

In the various embodiments, a cleansing composition according to the disclosure includes at least one polyurethane, wherein the polyurethane is a water-based dispersion (referred to herein alternatively as polyurethane and water-based dispersion of polyurethane). Suitable water-based dispersions of polyurethane for use in accordance with the disclosure are those water-based polyurethane dispersions that are typically employed to function as a film former or plasticizer for personal care products such as mask, mascara, eyeliner, sunscreen & hair-styling.

Accordingly, in at least some embodiments, the composition according to the disclosure includes a polyurethane selected from the group consisting of Polyurethane-62 (And) Trideceth-6, Polyurethane-6, Polyurethane-34, Polyurethane-35, Polyurethane-39, and combinations thereof.

In some particular embodiments, the polyurethane is Polyurethane-62 (And) Trideceth-6.

In the various embodiments, the water-based dispersion of polyurethane is present in a cleansing composition, by weight, in an amount in the range of at least 0.2%, or about 0.3% to about 10%, or up to 10%, or about 0.2% to about 1%, or about 0.2 to about 0.8%, or about 0.3% to about 0.5%, or about 0.4%, or about 1% to about 5%, or about 5% to about 10%, or any value, range, or sub-range therebetween. One of ordinary skill in the art, however, will appreciate that other ranges are within the scope of the invention.

Thus, one or more polyurethane is present by weight, based on the total weight of the composition, from about, 0.2, 0.3, 0.4, 0.5, 0.6, 0.7, 0.8, 0.9, 11, 2, 3, 4, 5, 6, 7, 8, 9 to about 10 percent, including increments and ranges therein and there between.

In the composition as exemplified herein, the raw material forms of the polyurethanes are provide with the following percentages of active ingredient in the raw material formulation: Polyurethane-62 (And) Trideceth-6 (90% active), Polyurethane-6 (30% active), Polyurethane-34 (32% active), Polyurethane-35 (41% active), and Polyurethane-39 (20% active). The active percentages shown in the examples herein represent the amount of active used in the composition, not the amount of RM, unless otherwise stated.

Silicone Oil-In-Water Microemulsion

In various embodiments, the cleansing composition includes one or more silicone microemulsion components as silicone oil-in-water microemulsions. The silicone oil-in-water microemulsion component comprises (a) water (˜40%), (b) silicone (˜55%) and the remainder surfactant to make an emulsion with micelles/droplets having a diameter in the range from about 110 nm to about 1,000 nm. Reference may be made to U.S. Pat. No. 6,071,975 which describes methods of preparing silicone oil-in-water microemulsions from which some nonlimiting examples of microemulsions that may be used according to this disclosure.

In some particular embodiments, useful silicone oil-in-water microemulsions include, but are not limited to: amodimethicone (and) trideceth-5 (and) amodimethicone (and) trideceth-10; water (and) amodimethicone (and) trideceth-6; Amodimethicone (and) Cetrimonium Chloride (and) Trideceth-12; Trideceth-9 PG Amodimethicone (and) Trideceth-12; Amodimethicone (and) C11-15 Pareth-7 (and) Laureth-9 (and) Glycerin (and) Trideceth-12; amodimethicone (and) trideceth-6 (and) cetrimonium chloride; dimethicone (and) amodimethicone (and) trideceth-10 (and) peg-100 stearate (and) steareth-6 (and) trideceth-3; and combinations thereof. The foregoing are non-limiting examples of silicone oil-in-water microemulsions that may be used in accordance with the invention.

In some particular embodiments, the silicone oil-in-water microemulsions comprise amodimethicone (and) trideceth-5 (and) amodimethicone (and) trideceth-10.

In some embodiments, the silicone oil-in-water microemulsion are present in a cleansing composition, by weight, in an amount in the range of at least 0.2%, or about 0.3% to about 10%, or up to 10%, or about 0.2% to about 1%, or about 1% to about 5%, or about 2% to about 7%, or about 5% to about 10%, or any value, range, or sub-range therebetween. One of ordinary skill in the art, however, will appreciate that other ranges are within the scope of the invention.

Thus, any one of the one more silicone oil-in-water microemulsion components is present, by weight, based on the total weight of the composition, from about 0.2, 0.3, 0.4, 0.5, 0.6, 0.7, 0.8, 0.9, 11, 2, 3, 4, 5, 6, 7, 8, 9 to about 10 weight percent, including increments and ranges therein and there between.

Oil/Ester

A cleansing composition includes at least one oil. As used herein, oil refers to any nonpolar compound that is a liquid at 25° C. and is hydrophobic and lipophilic. Oil that is suitable for use herein may be volatile or non-volatile. The term “volatile oil” relates to oil that is capable of evaporating on contact with the skin or a keratin fiber in less than one hour, at room temperature and atmospheric pressure. The volatile oil(s) are liquid at room temperature and have a non-zero vapor pressure, at room temperature and atmospheric pressure, ranging in particular from 0.13 Pa to 40 000 Pa (10-3 to 300 mmHg). The term “non-volatile oil” relates to oil which remains on the skin or the keratin fiber, at room temperature and atmospheric pressure, for at least several hours and which in particular has a vapor pressure of less than 10-3 mmHg (0.13 Pa).

The term “silicone oil” relates to oil comprising at least one silicon atom, and especially at least one Si—O group. The term “fluoro oil” relates to oil comprising at least one fluorine atom. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil or a synthetic hydrocarbon-based oil. Further, suitable oil may be a mineral hydrocarbon-based oil, a plant hydrocarbon-based oil, or a synthetic hydrocarbon-based oil.

Silicone Oils

The cleansing composition may comprise one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the cosmetic composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Other examples of silicone oils that may be mentioned include volatile linear or cyclic silicone oils, especially those with a viscosity 8 centistokes (8×106 m2/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Fluoro Oils

The cleansing composition may comprise one or more fluoro oils. For example, the one or more fluoro oil may be selected from the group consisting of perfluoromethylcyclopentane, perfluoro-1,3-dimethylcyclohexane, dodecafluoropentane, tetradecafluorohexane, bromoperfluorooctyl, nonafluoromethoxybutane, nonafluoroethoxyisobutane and 4-trifluoromethylperfluoromorpholine. Volatile fluoro oils, such as nonafluoromethoxybutane, decafluoropentane, tetradecafluorohexane, dodecafluoropentane, may also be used.

Hydrocarbon-Based Oils

The cleansing composition may comprise one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the composition may include a synthetic oil selected from the group consisting of hydrogenated polyisobutene and hydrogenated polydecene. A hydrocarbon-based oil may be a non-volatile hydrocarbon-based, such as:

(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.

(ii) synthetic ethers containing from 10 to 40 carbon atoms;

(iii) linear or branched hydrocarbons of mineral or synthetic origin, such as petroleum jelly, polydecenes, hydrogenated polyisobutene such as Parleam, and 40 squalane;

(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI® by Enichem Augusta Industriale, and also di(linear C14-C15 alkyl) tartrates, such as those sold under the name Cosmacol ETL® by the same company; or acetates;

(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;

(vi) higher fatty acids, such as oleic acid, linoleic acid or linolenic acid;

(vii) carbonates, such as dicaprylyl carbonate, such as the product sold under the name Cetiol CC® by Cognis;

(viii) fatty amides, such as isopropyl N-lauroyl sarcosinate, such as the product sold under the trade name Eldew SL 205® from Ajinomoto; and

(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

Hydrocarbon-based oils may be glyceride triesters and in particular to caprylic/capric acid triglycerides, synthetic esters and in particular isononyl isononanoate, oleyl erucate, C12-C15 alkyl benzoate, 2-ethylphenyl benzoate and fatty alcohols, such as octyldodecanol. As volatile hydrocarbon-based oils, mention is made of hydrocarbon-based oils containing from 8 to 16 carbon atoms and in particular of branched C8-C16 alkanes, such as C8-C16 isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C C8-C16 esters, and isohexyl neopentanoate.

In some embodiments, the cleansing composition may comprise one or more oils such as from those described herein above, and from oils that may be selected from branched or linear, liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C11, C12, C13, C14, C15, C16, C17, C18 to C19. In some particular embodiments, suitable liquid alkanes that may be used according to the disclosure include hydrocarbon-based oils containing from 8 to 16 carbon atoms, and especially branched C8-C16 alkanes such as C8-C16 isoalkanes.

In some embodiments, the cleansing composition may comprise one or more oils selected from polar emollients selected from esters, triglycerides, ethers, carbonates, alcohols, oils, butters, fatty acids, and their combinations thereof. In various embodiments, the polar emollients may be selected from those with a molecular weight of 400 g/mol or less. More, generally, the polar emollient may have a molecular weight in the range from about 50 g/mol to about 350 g/mol.

In some embodiments, suitable polar emollients that may be used according to the disclosure include those derived from C12-050 fatty acids, preferably C16-C22 saturated fatty acids, and monohydric alcohols. In some embodiments, such esters may be chosen from isopropyl myristate, methyl palmitate, isopropyl laurate, isopropyl palmitate, ethylhexyl palmitate, ethylhexyl laurate, ethylhexyl oleate, ethylhexyl isononanoate, myristyl myristate, 2-ethylhexyl caprate/caprylate (or octyl caprate/caprylate), 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate and their mixtures.

In some embodiments, the at least one oil comprises one of isopropyl myristate, dicaprylyl ether, ethylhexyl palmitate, isopropyl palmitate, cetearyl ethylhexanoate, isononyl isononanoate, isopropyl isostearate, diisopropyl sebacate, coco caprylate/caprate, diisopropyl adipate, and combinations thereof. In some particular embodiments the at least one lipophilic emollient comprises isopropyl myristate.

In some embodiments, appropriate oils and esters may include, but are not limited to, Helianthus annuus (sunflower) seed oil, isohexadecane, palmitic acid, lauric acid, myristic acid, stearic acid, PEG-150 distearate, isopropyl myristate, ethylhexyl palmitate, sorbitan oleate or other certain hydrocarbons, fatty acids, or esters. In some other embodiments, appropriate oils may include, but are not limited to, castor oil, coconut oil, palm oil, argan oil, jojoba oil, and combinations thereof. in some other embodiments, appropriate esters may include, but are not limited to, C15-19 alkane, neopentyl glycol diethylhexanoate, cetyl ethylhexanoate, isostearyl hydroxystearate, isodecyl neopentanoate, triisostearyl trilinoleate, triisostearyl citrate, and combinations thereof.

In some particular embodiments, as exemplified herein, the at least one oil or ester comprises one or more of isohexadecane, Helianthus annuus seed oil, palmitic acid, lauric acid, myristic acid, stearic acid, PEG-150 distearate, and combinations thereof. In some embodiments, the at least one oil or ester comprises each of isohexadecane, Helianthus annuus seed oil, palmitic acid, lauric acid, myristic acid, PEG-150 distearate, and stearic acid.

In some embodiments, the oil or ester is present in a cleansing composition, by weight, in an amount of at least 0.1%, or about 0.1% to about 50%, or up to 50%, or about 0.1% to about 1%, or about 1% to about 4%, or about 4% to about 10%, or about 10% to about 15%, or about 20% or more, or not more than about 50%, or any value, range, or sub-range therebetween. One of ordinary skill in the art, however, will appreciate that other ranges are within the scope of the invention.

Thus, each of at least one or more oils or a combination thereof in the composition may be present by weight, based on the total weight of the composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 120, 21, 22, 23, 24, 25, 26, 27, 28, 29, 30, 3, 32, 33, 34, 35, 36, 37, 38, 39, 40, 41, 42, 43, 44, 45, 46, 47, 48, 49 to about 50 percent, including increments and ranges therein and there between.

Surfactants

In some embodiments, a cleansing composition may include at least one surfactant. The surfactant preferably is high stability and high foaming.

In accordance with various embodiments, any of a variety of surfactants may be employed, including, without being limiting, sodium C14-16 olefin sulfonate, disodium laureth sulfosuccinate (and) sodium lauryl sulfoacetate, sodium lauroyl sarcosinate, sodium methyl cocoyl taurate, sodium methyl lauroyl taurate, sodium cocoyl alaninate, sodium cocoyl isethionate, cocamidopropyl hydroxysultaine, cocamidopropyl betaine, coco-betaine, sodium cocoyl glycinate, sodium lauroyl glutamate, potassium cocoyl glycinate, decyl glucoside, lauryl glucoside, and combinations thereof.

In some particular embodiments, the composition includes anionic surfactants selected from the group consisting of sodium C14-16 olefin sulfonate, disodium laureth sulfosuccinate (and) sodium lauryl sulfoacetate, sodium lauroyl sarcosinate, and combinations thereof. In an exemplary embodiment, the composition includes sodium C14-16 olefin sulfonate.

In some embodiments, the surfactant is present in a cleansing composition, by weight, in an amount in the range of at least 1%, or about 1% to about 50%, or up to 50%, or about 1% to about 5%, or about 3% to about 8%, or about 6%, or about 5% to about 10%, or about 7% to about 15%, or about 10% to about 30%, or about 30% to about 50%, or any value, range, or sub-range therebetween. One of ordinary skill in the art, however, will appreciate that other ranges are within the scope of the invention.

Thus, any water is present, by weight, based on the total weight of the composition, from about 1, 2, 3, 4, 5, 6, 7, 8, 9, 10, 11, 12, 13, 14, 15, 16, 17, 18, 19, 20, 21, 22, 23, 23, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

Water-Based Solvents

A cleansing composition includes at least one solvent comprising at least water.

Water

In the various embodiments, water is present in a cleansing composition, by weight, in an amount from at least 20%, or about 20% to about 50%, or up to 50%, or about 20% to about 30%, or about 30% to about 40%, or about 40% to about 50%, or any value, range, or sub-range therebetween. One of ordinary skill in the art, however, will appreciate that other ranges are within the scope of the invention.

Thus, any water is present, by weight, based on the total weight of the composition, from about 20, 21, 22, 23, 23, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

Water-Soluble Solvents

In accordance with some embodiments, the cleansing composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), polyols, glycols, and combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and combinations thereof.

In some cases, a water-soluble solvent may be selected from the group consisting of one or more glycols, C1-4 alcohols, glycerin, and combinations thereof.

In accordance with the various embodiments total amount of the at least one water-soluble solvent, when present, may vary, is from about 0.5% to about 25%, or from about 0.5% to about 20%, or from about 1% to about 20%, or from about 1% to about 10%, or from about 2% to about 5% or any suitable combination, sub-combination, range, or sub-range thereof by weight, based on the weight of the composition. One of ordinary skill in the art, however, will appreciate that other ranges are within the scope of the invention.

Thus, any one water-soluble solvent, when present, is present, by weight, based on the total weight of the composition, from about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

It will be appreciated by a skilled artisan that any non-water solvents are present only to the extent and in amounts that do not materially adversely affect the basic and novel characteristic(s) of the claimed disclosure. Thus, in some embodiments that include preservatives, such preservatives will not materially adversely affect the cleansing composition forming a stable gel.

Humectant/Hydrating Agent

In accordance with the disclosure, one or more humectants may be present in the composition. In some embodiments, the humectant may comprise one or more of polyols, including, for example, glycerin, glycerol, glycols, such as, caprylyl glycol, butylene glycol, propylene glycol, isoprene glycol, dipropylene glycol, hexylene glycol and polyethylene glycols, monoethylene glycol, diethylene glycol, triethylene glycol, diethylene glycol, hexylene glycol, glycol ethers, such as, monopropylene, dipropylene and tripropylene glycol alkyl(C1-C4)ethers, squalane, triacetin, sugars, such as, glucose, xylitol, maltitol, sorbitol, sucrose pentaerythritol, inositol, pyrrolidone carboxylic acid, lactic acid, lithium chloride, acetamide MEA, sodium lactate, urea, dicyanamide, hyaluronic acid, aloe vera, honey, and seaweed extract.

In some particular embodiments, as exemplified herein, a suitable humectant comprises glycerin and sorbitol.

In accordance with the various embodiments, the amount of humectant present in the composition can range from about 1% to about 10%, or from about 1% to about 8%, or from about 1% to about 5%, or from about 2% to about 3%, or any suitable combination, sub-combination, range, or sub-range thereof by weight, based on the weight of the composition. One of ordinary skill in the art, however, will appreciate that other ranges are within the scope of the invention. One of ordinary skill in the art, however, will appreciate that other ranges are within the scope of the invention.

Thus, any one of or a combination of humectant, when present, may be present, by weight, based on the total weight of the composition, is from about 1, 2, 3, 4, 5, 6, 7, 8, 9, to about 10 weight percent, including increments and ranges therein and there between.

Preservatives

In accordance with the disclosure, one or more preservatives and/or antimicrobials may be present in the composition. Any preservative commonly used in cosmetic cleansing compositions is an acceptable preservative for the cleansing compositions herein, such as phenoxyethanol, members from the paraben family such as the methyl, ethyl, propyl, butyl or isobutyl parabens, 4-hydroxy benzoic acid, benzoic acid, sorbic acid, dehydroacetic acid, triclosan, benzyl alcohol, chlorophenesin, or salicylic acid, for example. At more concentrated amounts of suitable solvents for optional additives, in particular, suitable solvents for antimicrobials and preservatives, members from the paraben family may be used as a preservative.

In some embodiments, the preservative may comprise one or more of preservatives selected from the group consisting of organic acids, parabens, formaldehyde donors, phenol derivatives, quaternary ammoniums, alcohols, isothiazolinones, and combinations thereof. Preservatives having antibacterial activity are optionally present in the cleansing compositions of the present invention. Examples of organic acid preservatives include, but are not limited to, sodium benzoate, potassium sorbate, benzoic acid and dehydroacetic acid, sorbic acid, and combinations thereof. A preferred organic acid preservative system includes a mixture of sodium benzoate and potassium sorbate. Examples of paraben preservatives include, but are not limited to, alkyl para-hydroxybenzoates, wherein the alkyl radical has from 1, 2, 3, 4, 5 or 6 carbon atoms and for example, from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben). Examples of formaldehyde donor preservatives include, but are not limited to, 1,3-Dimethylol-5,5-dimethylhydantoin (DMDM hydantoin), imidazolidinyl urea, gluteraldehyde, and combinations thereof. Examples of quaternary ammonium preservatives include, but are not limited to, benzalkonium chloride, methene ammonium chloride, benzethonium chloride, and combinations thereof. Examples of alcohol preservatives include, but are not limited to, ethanol, benzyl alcohol, dichlorobenzyl alcohol, phenoxyethanol, and combinations thereof. Examples of isothiazolone preservatives include, but are not limited to, methylchloroisothiazolinone, methylisothiazolinone, and combinations thereof.

In some particular embodiments, as exemplified herein, appropriate preservatives include, but are not limited to, phenoxyethanol.

In some embodiments, the preservative includes one or more preservatives, the one or combination present at a concentration, by weight of about 0.001% to about 5%, or alternatively about 0.05% to about 2.5% or alternatively about 0.1% to about 2.0%, based upon weight of the composition. One of ordinary skill in the art, however, will appreciate that other ranges are within the scope of the invention.

Thus, any one of or a combination of preservatives, when present, may be present, by weight, based on the total weight of the composition, is from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 1.0, 1.1, 1.2, 1.3, 1.4, 1.5, 1.6, 1.7, 1.8, 1.9, 2, 3, 4, to about 5 weight percent, including increments and ranges therein and there between.

It will be appreciated by a skilled artisan that any preservatives are present only to the extent and in amounts that do not materially adversely affect the basic and novel characteristic(s) of the claimed disclosure. Thus, in some embodiments that include preservatives, such preservatives will not materially adversely affect the cleansing composition forming a stable gel.

Optional Additives

In some embodiments, a cleansing composition includes at least one additive used in the cosmetics field which does not affect the properties of the composition according to the invention, such as, fragrances, pH adjusters (citric acid, sodium chloride; neutralizing or pH-adjusting agents (e.g., triethylamine (TEA) and sodium hydroxide), and combinations thereof), other cosmetically acceptable additives, such as but not limited to, pearlescent agents, silica, and coloring materials; essential oils; fruit extracts, for example, Pyrus Malus (Apple) Fruit Extract, and Aloe Barbadensis Leaf Juice Powder. Although the optional additives are given as examples, it will be appreciated that other optional components compatible with cosmetic applications known in the art may be used.

In accordance with the various embodiments, the amount of one or more additives, alone or in combination, present in the composition can be present in the composition according to the disclosure in a range from about 0.001% to about 20%, by weight, or from about 0.005% to about 0.01%, or from about 0.01% to about 0.1%, or from about 0.15% to about 5%, or from about 0.40% to about 4%, or from about 0.5% to about 2.5% by weight, or from about 1% to about 2%, or any suitable combination, sub-combination, range, or sub-range thereof by weight, based on the total weight of the composition. One of ordinary skill in the art, however, will appreciate that other ranges are within the scope of the invention.

Thus, any one or a combination of additives may be present, by weight, based on the total weight of the composition, each one or the combination present from about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.20, 0.30, 0.40, 0.50, 0.60, 0.70, 0.80, 0.90, 1.0, 2, 3, 4, 5, 6, 7, 8, 9, 10, 11, 12, 13, 14, 15, 16, 17, 18, 19 to about 20 weight percent, including increments and ranges therein and there between.

Although the optional additives are given as examples, it will be appreciated that other optional components compatible with cosmetic applications known in the art may be used that are suitable. It will be appreciated by a skilled artisan that any optional additives are present only to the extent and in amounts that do not materially adversely affect the basic and novel characteristic(s) of the claimed disclosure. Thus, in some embodiments that include optional additives, such optional additives will not materially adversely affect the cleansing composition forming a stable gel.

### Examples

The following examples are intended to further illustrate the present disclosure. They are not intended to limit the disclosure in any way. Unless otherwise indicated, all parts are by weight.

Compositions

Generally, comparative benchmark compositions known in the art include: Clinique Rinse-Off Foaming Cleanser—only good foaming, but can't remove long lasting makeup products; Neutrogena One Step Gentle Cleanser—low foaming, and can't remove long lasting makeup products; Neutrogena Oil-Free Eye Makeup Remover—No foaming; and Cetaphil Gentle Waterproof Makeup Remover—No foaming with makeup removal ability.

TABLE 1 lists ingredients and their amounts in weight percentage in Inventive Example 1 as representative embodiments according to the disclosure.

TABLE 1
Inventive Composition

| | Inventive |
|---|---|
| INCI UE Active | Composition |
| SURFACTANT (e.g., SODIUM C14-16 OLEFIN | ~3-8 |
| SULFONATE**) | |
| POLYURETHANE (e.g., POLYURETHANE-62 (and) | ~0.3-0.6 |
| TRIDECETH-6*) | |
| PRESERVATIVE | ~0.5-2.5 |
| HUMECTANT (e.g., GLYCERIN, SORBITOL, etc) | ~1-15 |
| OIL (ISOHEXADECANE) | ~8-20 |
| WATER | QS |
| ESTER (e.g., PALMITIC ACID, LAURIC ACID, | ~0.5-10/each |
| MYRISTIC ACID, STEARIC ACID, PEG-150 | |
| DISTEARATE) | |
| LAURIC ACID | ~0.5-10 |
| POTASSIUM HYDROXIDE | ~0.5-10 |
| MYRISTIC ACID | ~0.5-10 |
| STEARIC ACID | ~0.5-2.5 |
| PEG-150 DISTEARATE | ~0.5-5 |
| OIL (HELIANTHUS ANNUUS SEED OIL) | ~1-15 |
| SI EMULSION (e.g., AMODIMETHICONE (and) | ~2-8 |
| TRIDECETH-5 (and) AMODIMETHICONE (and) | |
| TRIDECETH-10, | |
| DIVINYLDIMETHICONE/DIMETHICONE | |
| COPOLYMER (and) C12-13 PARETH-3 | |
| (and) C12-13 PARETH-23, AMODIMETHICONE | |
| (and) C11-15 PARETH-7 (and) LAURETH-9 | |
| (and) GLYCERIN (and) TRIDECETH-12) | |

*RM: Polyurethane-62 (And) Trideceth-6 RAW MATERIAL = 90% PU in carrier
**RM: SODIUM C14-16 OLEFIN SULFONATE raw material = 40% surfactant active in carrier

In a first representative example (Inventive 1), the composition, for example as provided according to Table 1, includes about 0.4% polyurethane, about 5% silicone emulsion comprising AMODIMETHICONE (and) TRIDECETH-5 (and) AMODIMETHICONE (and) TRIDECETH-10, about 10% esters (including palmitic acid, lauric acid, myristic acid, stearic acid, PEG-150 distearate), about 10% Helianthus annuus SEED OIL, about 20% isohexadecane, about 6% surfactant, less than 1% preservative, about 11% humectant, and about 5% pH adjuster, the balance water.

In a second representative example (Inventive 2), the composition, for example as provided according to Table 1, includes about 0.4% polyurethane, about 5% silicone emulsion comprising DIVINYLDIMETHICONE/DIMETHICONE COPOLYMER (and) C12-13 PARETH-3 (and) C12-13 PARETH-23, about 10% esters (including palmitic acid, lauric acid, myristic acid, stearic acid, PEG-150 distearate), about 10% Helianthus annuus SEED OIL, about 20% isohexadecane, about 6% surfactant, less than 1% preservative, about 11% humectant, and about 5% pH adjuster, the balance water.

In a third representative example (Inventive 3), the composition, for example as provided according to Table 1, includes about 0.4% polyurethane, about 5% silicone emulsion comprising, AMODIMETHICONE (and) C11-15 PARETH-7 (and) LAURETH-9 (and) GLYCERIN (and) TRIDECETH-12, about 10% esters (including palmitic acid, lauric acid, myristic acid, stearic acid, PEG-150 distearate), about 10% Helianthus annuus SEED OIL, about 20% isohexadecane, about 6% surfactant, less than 1% preservative, about 11% humectant, and about 5% pH adjuster, the balance water.

In a fourth representative example (Inventive 4), the composition, for example as provided according to Table 1, includes about 0.4% polyurethane, about 5% silicone emulsion comprising AMODIMETHICONE (and) TRIDECETH-5 (and) AMODIMETHICONE (and) TRIDECETH-10, about 10% esters (including palmitic acid, lauric acid, myristic acid, stearic acid, PEG-150 distearate), about 1% Helianthus annuus SEED OIL, about 8% isohexadecane, about 6% surfactant, less than 1% preservative, about 11% humectant, and about 5% pH adjuster, the balance water.

TABLE 2 lists the ingredients and their amounts in weight percentage in Comparative Composition Examples 1-3.

TABLE 2
Comparative Compositions

| | Comparative | Comparative | Comparative |
|---|---|---|---|
| INCI UE Active | 1 | 2 | 3 |
| SODIUM C14-16 | ~6 | ~6 | ~6 |
| OLEFIN SULFONATE** | | | |
| POLYURETHANE-62 | ~0.3 | 0 | ~0.4 |
| (AND) TRIDECETH-6* | | | |
| PRESERVATIVE | <1 | <1 | <1 |
| HUMECTANTS | ~10 | ~10 | ~10 |
| HYDROXYSTEARIC | ~1 | ~2 | |
| ACID | | | |
| ISOHEXADECANE | 25.0 | 20.0 | 20.0 |
| WATER | QS | QS | QS |
| PALMITIC ACID | <1 | ~1 | ~1 |
| LAURIC ACID | ~5 | ~5 | ~5 |
| POTASSIUM | 5.0 | 4.5 | 4.5 |
| HYDROXIDE | | | |
| MYRISTIC ACID | ~4 | ~4 | ~4 |
| STEARIC ACID | ~1 | ~1 | ~1 |
| HELIANTHUS ANNUUS | | 5.0 | 5.0 |
| SEED OIL | | | |
| GLYCERYL STEARATE | | | ~3 |

*RM: Polyurethane-62 (And) Trideceth-6 RAW MATERIAL = 90% PU in carrier
**RM: SODIUM C14-16 OLEFIN SULFONATE raw material = 40% surfactant active in carrier

Inventive and Comparative compositions were evaluated for pigmented makeup removal efficacy. The results of the evaluations are shown in TABLE 3.

As used herein with respect to makeup removal, the term “Makeup Removal Capacity,” or “MUR Capacity” means and refers to the capacity of a composition (inventive or comparative) to remove a long lasting makeup, such as a pigmented lipstick or mascara, as exemplified herein, or other makeup product that is designed to have long lasting staying properties with resistance to removal and smudging. The data in TABLE 3 reflect removal results with mascara and lipstick formulations. All percentages shown pertain to mascara removal, where the percentages reflect the % of makeup removed with the tested formulation, which is determined based on the weight of mascara removed (g) divided by the weight of the mascara applied (g) multiplied by 100. Other representations of makeup removal capacity may be characterized on a scale from very low (˜0-25%), low (˜25-40%), middle (˜40-55%), high (˜55-85%) to very high (>85%).

TABLE 3
Evaluation of Long Lasting Pigmented Makeup Removal

| | | | MAKEUP |
|---|---|---|---|
| | | | REMOVAL |
| | | | CAPACITY |
| | | | (wt % |
| | | | mascara |
| FLA | POLYURETHANE ^{#} | SILICONE EMULSION | removed**) |
| Comparative 1 | POLYURETHANE-62 | NO | 45.22 |
| (Base) | (and) TRIDECETH-6 | | |
| Comparative 2 | NO | NO | 20.35 |
| NO PU | | | |
| Comparative 1 | POLYURETHANE-6 | NO | 26.69 |
| varied PU | | | |
| Comparative 1 | POLYURETHANE-34 | NO | 28.36 |
| varied PU | | | |
| Comparative 1 | POLYURETHANE-35 | NO | 26.35 |
| varied PU | | | |
| Comparative 1 | POLYURETHANE-39 | NO | 24.58 |
| varied PU | | | |
| Inventive 1 | POLYURETHANE-62 | AMODIMETHICONE (and) | 80.25 |
| | (and) TRIDECETH-6 | TRIDECETH-5 (and) | |
| | | AMODIMETHICONE (and) | |
| | | TRIDECETH-10 | |
| Inventive 2 | POLYURETHANE-62 | DIVINYLDIMETHICONE/DIMETHICONE | 77.62 |
| | (and) TRIDECETH-6 | COPOLYMER (and) C12-13 | |
| | | PARETH-3 (and) C12-13 PARETH-23 | |
| Inventive 3 | POLYURETHANE-62 | AMODIMETHICONE (and) C11-15 | 73.52 |
| | (and) TRIDECETH-6 | PARETH-7 (and) LAURETH-9 (and) | |
| | | GLYCERIN (and) TRIDECETH-12 | |
| Benchmark | NO | NO | 9.62 |
| Commercial | | | |
| Sulfate-Free | | | |
| Foaming Cleanser | | | |
| Comparative 3 | POLYURETHANE-62 | NO | 44.7 |
| (O/W Emulsion | (and) TRIDECETH-6 | | |
| with PEG-150 | | | |
| Distearate AND | | | |
| SUNFLOWER | | | |
| OIL) | | | |

^{#} = comparisons based on an equivalent amount of PU active in each tested formulation; see above for amounts of RM in various PU formulations
**Long Lasting Makeup, Mascara: Maybelline Colossal Big Shot mascara (VP/eicosene copolymer, 3.3% allyl stearate/VA copolymer, 1-ethylenediamine/stearyl dimer dilinoleate copolymer, wax)

Inventive and Comparative compositions were evaluated for foaming property. The results of the evaluations are shown in TABLE 4.

TABLE 4 provides results of Foaming Capacity and the Makeup Removal Capacity for various tested formulations (Foaming Capacity references the scale described herein above).

TABLE 4
Tabulation of Foaming and Makeup Removal Capacities

| | | | | MAKEUP |
|---|---|---|---|---|
| | | SILICONE | FOAMING | REMOVAL |
| FLA | POLYURETHANE | EMULSION | CAPACITY | CAPACITY |
| Comparative 1 (base) | POLYURETHANE-62 | NO | Very High | Middle |
| | (and) TRIDECETH-6 | | | |
| Comparative 2 NO PU | NO | NO | Very Low | Very Low |
| Comparative 1 Base varied PU | POLYURETHANE-6 | NO | Middle | Low |
| Comparative 1 Base varied PU | POLYURETHANE-34 | NO | High | Low |
| Comparative 1 Base varied PU | POLYURETHANE-35 | NO | Low | Low |
| Comparative 1 Base varied PU | POLYURETHANE-39 | NO | Middle | Low |
| Inventive 1 | POLYURETHANE-62 | YES* | Very High | High |
| | (and) TRIDECETH-6 | | | |
| Inventive 2 | POLYURETHANE-62 | YES* | Very High | High |
| | (and) TRIDECETH-6 | | | |
| Inventive 3 | POLYURETHANE-62 | YES* | Very High | High |
| | (and) TRIDECETH-6 | | | |
| Benchmark Commercial | NO | NO | Middle | Very Low |
| Sulfate-Free Foaming | | | | |
| Cleanser | | | | |
| Comparative 3 | POLYURETHANE-62 | NO | Very High | Middle |
| | (and) TRIDECETH-6 | | | |
| Comparative Silicone | NO | YES (10%)* | None | High |
| Emulsion with surfactant | | | | |
| Comparative | NO | YES (10%)* | None | Very High |
| Silicone Emulsion Alone | | | | |

*see Table 3

Referring to each of TABLES 3 and 4, it is evident that only the inventive composition that includes each of the water dispersed polyurethane, silicone oil-in-water microemulsion and oil/ester (exemplified as a blend of oils/esters) demonstrates high Foaming Capacity and high Makeup Removal Capacity. This result is unexpected insofar as typical makeup removal formulations that include both oil/ester solvents with surfactants and/or silicon emulsions do not typically provide any foaming, and further insofar as typical foaming-type formulations that include polyurethane do not achieve significant makeup removal and are known to lose foaming capabilities upon addition of oils in high amounts such as are required for hair cleansers. The instant inventive composition provides high foaming with high makeup removal capabilities.

While the disclosure has been described with reference to a preferred embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The articles “a” and “an”, as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the composition, typically 0% by weight, based on the total weight of the composition. The term “essentially free” means that, while it prefers that no excluded material is present in the composition, it is possible to have very small amounts of the excluded material in the composition of the invention, provided that these amounts do not materially affect the advantageous properties of the composition. In particular, “essentially free” means that excluded material can be present in the composition at an amount of less than about 0.1% by weight, based on the total weight of the composition.

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The examples serve to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed is:

1. A cleansing composition comprising:
at least one polyurethane, wherein the at least one polyurethane is water-based;
a silicone oil-in-water microemulsion; and
at least one oil or ester.

2. The cleansing composition of claim 2, wherein the at least one polyurethane is present in an amount of 0.2 wt % to 10 wt % of the cleansing composition.

3. The cleansing composition of claim 1, wherein the at least one polyurethane is selected from the group consisting of polyurethane-62 (and) trideceth-6, polyurethane-34, polyurethane-35, polyurethane-39, polyurethane-6, and combinations thereof

4. The cleansing composition of claim 1, wherein the at least one polyurethane comprises polyurethane-62 (and) trideceth-6.

5. The cleansing composition of claim 1, wherein the at least one silicone oil-in-water microemulsion is present in an amount of 0.2 wt % to 10 wt % of the cleansing composition.

6. The cleansing composition of claim 1, wherein the at least one silicone oil-in-water microemulsion is selected from the group consisting of amodimethicone (and) trideceth-5 (and) amodimethicone (and) trideceth-10; WATER (and) AMODIMETHICONE (and) TRIDECETH-6; Amodimethicone (and) Cetrimonium Chloride (and) Trideceth-12; Trideceth-9 PG Amodimethicone (and) Trideceth-12; Amodimethicone (and) C11-15 Pareth-7 (and) Laureth-9 (and) Glycerin (and) Trideceth-12; amodimethicone (and) trideceth-6 (and) cetrimonium chloride; dimethicone (and) amodimethicone (and) trideceth-10 (and) peg-100 stearate (and) steareth-6 (and) trideceth-3; and combinations thereof.

7. The cleansing composition of claim 1, wherein the at least one silicone oil-in-water microemulsion comprises amodimethicone (and) trideceth-5 (and) amodimethicone (and) trideceth-10.

8. The cleansing composition of claim 1, wherein the at least one oil or ester is present in an amount of 0.5 wt % to 50 wt % of the cleansing composition.

9. The cleansing composition of claim 1, wherein the at least one at least one oil or ester is selected from the group consisting of a synthetic oil, a nature oil, an ester, and combinations thereof.

10. The cleansing composition of claim 1, wherein the at least one at least one oil or ester is selected from the group consisting of isohexadecane, Helianthus annuus seed oil, palmitic acid, lauric acid, myristic acid, stearic acid, PEG-150 distearate, and combinations thereof.

11. The cleansing composition of claim 1, comprising at least on surfactant selected from the group consisting of sodium C14-16 olefin sulfonate, disodium laureth sulfosuccinate (and) sodium lauryl sulfoacetate, sodium lauroyl sarcosinate and combinations thereof.

12. The cleansing composition of claim 11, wherein the at least one surfactant is present in the composition in an amount of 1 wt % to 50 wt % of the cleansing composition.

13. The cleansing composition of claim 11, wherein the at least one surfactant comprises sodium C14-16 olefin sulfonate.

14. A cleansing composition comprising:
at least one polyurethane, wherein the at least one polyurethane is water-based, the at least one polyurethane present in an amount of 0.2 wt % to 10 wt % of the cleansing composition;
at least one silicone oil-in-water microemulsion present in an amount of 0.2 wt % to 10 wt % of the cleansing composition; and
at least one oil or ester present in an amount of 0.5 wt % to 50 wt % of the cleansing composition.

15. The cleansing composition of claim 14, wherein the at least one polyurethane is selected from the group consisting of polyurethane-62 (and) trideceth-6, polyurethane-34, polyurethane-35, polyurethane-39, polyurethane-6, and combinations thereof.

16. The cleansing composition of claim 14, wherein the at least one silicone oil-in-water microemulsion is selected from the group consisting of amodimethicone (and) trideceth-5 (and) amodimethicone (and) trideceth-10; WATER (and) AMODIMETHICONE (and) TRIDECETH-6; Amodimethicone (and) Cetrimonium Chloride (and) Trideceth-12; Trideceth-9 PG Amodimethicone (and) Trideceth-12; Amodimethicone (and) C11-15 Pareth-7 (and) Laureth-9 (and) Glycerin (and) Trideceth-12; amodimethicone (and) trideceth-6 (and) cetrimonium chloride; dimethicone (and) amodimethicone (and) trideceth-10 (and) peg-100 stearate (and) steareth-6 (and) trideceth-3; and combinations thereof.

17. The cleansing composition of claim 14, wherein the at least one at least one oil or ester is selected from the group consisting of isohexadecane, Helianthus annuus seed oil, palmitic acid, lauric acid, myristic acid, stearic acid, PEG-150 distearate, and combinations thereof.

18. A cleansing composition comprising:
at least one polyurethane, wherein the at least one polyurethane is water-based, the at least one polyurethane present in an amount of 0.2 wt % to 0.5 wt % of the cleansing composition, and selected from the group consisting of polyurethane-62 (and) trideceth-6, polyurethane-34, polyurethane-35, polyurethane-39, polyurethane-6, and combinations thereof;
at least one silicone oil-in-water microemulsion present in an amount of 1 wt % to 7 wt % of the cleansing composition and selected from the group consisting of amodimethicone (and) trideceth-5 (and) amodimethicone (and) trideceth-10; WATER (and) AMODIMETHICONE (and) TRIDECETH-6; Amodimethicone (and) Cetrimonium Chloride (and) Trideceth-12; Trideceth-9 PG Amodimethicone (and) Trideceth-12; Amodimethicone (and) C11-15 Pareth-7 (and) Laureth-9 (and) Glycerin (and) Trideceth-12; amodimethicone (and) trideceth-6 (and) cetrimonium chloride; dimethicone (and) amodimethicone (and) trideceth-10 (and) peg-100 stearate (and) steareth-6 (and) trideceth-3; and combinations thereof;
at least one oil or ester present in an amount of 0.5 wt % to 5 wt % of the cleansing composition and selected from the group consisting of isohexadecane, Helianthus annuus seed oil, palmitic acid, lauric acid, myristic acid, stearic acid, PEG-150 distearate, and combinations thereof;
at least one surfactant present in an amount of 5 wt % to 15 wt % of the cleansing composition and selected from the group consisting of sodium C14-16 olefin sulfonate, disodium laureth sulfosuccinate (and) sodium lauryl sulfoacetate, sodium lauroyl sarcosinate and combinations thereof; and
water,
wherein the cleansing composition is a clear emulsion that exhibits high foaming upon rubbing on a substrate and removes high pigment oil based makeup.

19. The cleansing composition of claim 18 wherein the at least one polyurethane comprises polyurethane-62 (and) trideceth-6, the at least one silicone oil-in-water microemulsion comprises of amodimethicone (and) trideceth-5 (and) amodimethicone (and) trideceth-10, the at least one oil or ester comprises isohexadecane and Helianthus annuus seed oil, and the at least one surfactant comprises sodium C14-16 olefin sulfonate.

20. The cleansing composition of claim 18, wherein the amount of the at least one polyurethane is about 0.3 wt % to about 0.5 wt % of the cleansing composition, the amount of the at least one silicone oil-in-water microemulsion is about 2 wt % to about 8 wt % of the cleansing composition, the at least one oil or ester comprises a combination of oils and esters, the combination present in an amount from about 15 wt % to 25 wt % of the cleansing composition, and the amount of the at least one surfactant is about 10 wt % to about 20 wt %.
